(19) United States
(12) Patent Application Publication
(10) Pub. No.: US 2008/0070244 A1
(43) Pub. Date: Mar. 20, 2008

(54) Mold test kit

(75) Inventor: William J. Gartner, Paradise Valley, AZ (US)

Correspondence Address: QUARLES & BRADY LLP, 33 E. MAIN ST, SUITE 900, P.O BOX 2113, MADISON, WI, 53701-2113, US

(73) Assignee: Health Chek LLC

(21) Appl. No.: 11/521,003

(22) Filed: Sep. 14, 2006

(51) Int. Cl. C12Q 1/68

(52) U.S. Cl. 435/6

(57) ABSTRACT

The present invention provides a simple and easy-to-use mold detection kit for determining the presence or absence of airborne mold spores in multiple locations within a structure. The invention also provides methods for using the kit.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

Mold is a common allergen that can grow in many locations inside or outside a structure. It can also be found thriving inside building cavities, such as between walls. It is widely known that mold is also a very common indoor contaminant, which can cause illness. Molds reproduce by releasing spores into the air. The spores are extremely small, about 1 micron or about 0.00004 inches. Mold counts are often 1,000 times higher than pollen counts. Although tiny parts of a parent mold colony can break off and be inhaled, usually, the source of health problems is the inhalation of microscopic spores. Researchers have found that a person's allergic response is a biological reaction to the protein in mold, so a reaction can occur whether the inhaled spores are dead or alive. In addition, a thriving mold colony often releases various gases, including volatile organic compounds, that are a problem for sensitive individuals.

Different species of mold have different health effects ranging from mild symptoms to death. Some species of the mold Aspergillus can infect the entire body of a person, causing lung damage or other serious illnesses. For example, Histoplasma capsulatum can affect the lungs, but can also be systemic. A mold colony can use any organic material for food, and can even derive nutrition from a layer of dust on non-organic surfaces.

Accordingly, the first step in combating mold is to identify its presence by determining if toxic airborne mold spores are present in a home or an office. Historically, consumers have only had the options of hiring professional consultants to assess the mold in their homes. More recently, a limited selection of complicated home testing kits have come on the market. These home mold testing kits require too much work on the part of the consumer. For example, consumers have had to scrape walls, wear gloves and masks and fluff pillows and furniture, thereby creating ideal conditions for spores to become airborne.

Also, kits currently on the market can be expensive because they only allow testing of a single room per test kit. They are not designed to sample air in multiple rooms of a structure and therefore, require the purchase of several kits to survey an entire home or office. Indeed, some commercially available mold test kits clearly indicate on their packaging that the user should buy at least two kits to obtain a reliable result. Thus, an inexpensive and easy-to-use mold test kit for surveying the presence or absence of mold in multiple rooms within a home or office is desired.

### BRIEF SUMMARY OF THE INVENTION

The present invention is summarized as an inexpensive and easy-to-use mold detection kit for identifying the presence or absence of mold in several areas within a structure, such as a home or an office. The kit includes the following components: a plurality of culture plates for growing mold, wherein at least one of the plates is a reference plate to be placed outside of the structure to be tested; at least one container of mold growth medium; and instructions for using the kit components to facilitate mold detection wherein the components are packaged together and sold as a single unit. The kit also includes a mailer to be optionally used if further laboratory analysis of the culture plates is desired.

In one aspect, the kit can be used for detecting the presence or absence of mold within a structure such that multiple rooms or areas can be tested simultaneously.

In another aspect, the kit can be used to identify the presence or absence of mold in at least about forty-eight hours.

In a related aspect, the invention also provides a method for determining the presence or absence of airborne mold spores in multiple locations within a structure. The method includes providing a mold test kit having (i) a plurality of culture plates with lids for growing mold, wherein at least one of the plates serves as a reference plate, and (ii) at least one container of mold growth medium, wherein kit components (i) and (ii) are packaged together as a single unit; removing the lids from the plates; pouring mold growth medium into each plate; placing the plates in a variety of locations within a structure; wherein at least one plate is placed outside of the structure (i.e., preferably out doors) as an air control reference plate; running an indoor heating and/or a cooling system for at least about one hour; covering all culture plates with the corresponding lids and labeling each plate; placing all plates in a dark, warm location for at least about 48 hours; and visually comparing the reference plate with the indoor (test) plates for the presence of mold growth. The method also includes the option of re-inspecting the plates for mold growth after at least about 72 hours; and if desired submitting the culture plates for laboratory analysis.

Other objects advantages and features of the present invention will become apparent from the following specification.

### BRIEF DESCRIPTION OF THE SEVERAL VIEWS OF THE DRAWINGS

FIG. 1A-C illustrates the method for using the indoor mold test kit. (A) One plate is used to test for naturally occurring outdoor mold and one to three culture plates are placed at different locations indoors. (B) The outdoor reference plate is visually compared to the indoor plate(s) to determine the presence of mold. (C) Optionally, the plates may be sent for lab analysis if there is a significant difference between the outdoor reference plate and the indoor plate(s).

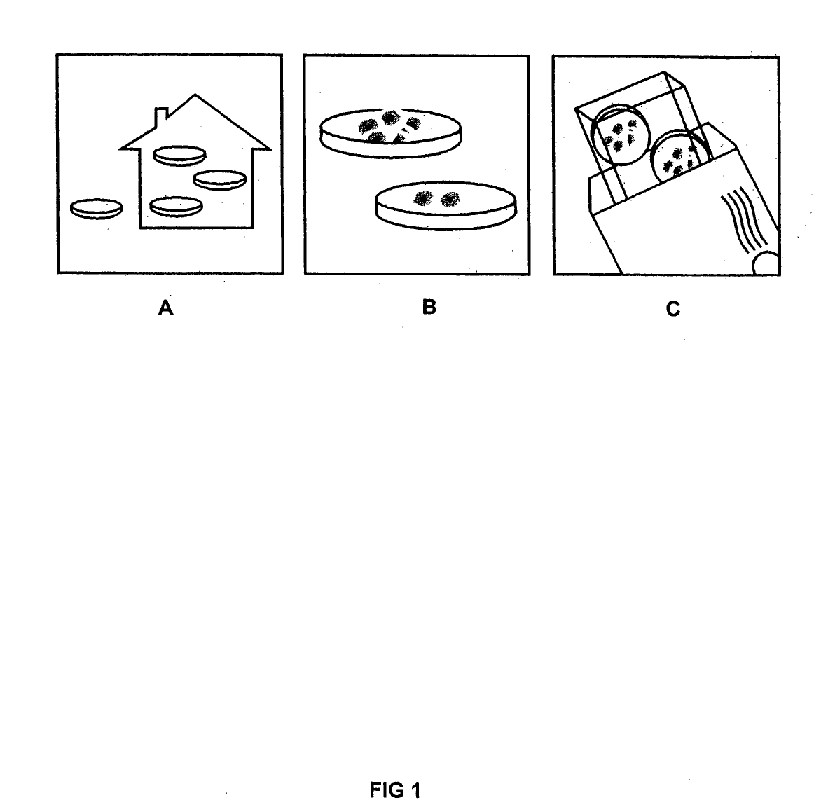

### DETAILED DESCRIPTION OF THE INVENTION

The present invention relates to an inexpensive and easy-to-use mold detection kit for identifying the presence or absence of mold in several areas within a structure, such as a home or office. In one embodiment, the kit includes a plurality of culture plates for growing mold, such that at least one of the plates serves as a reference plate; at least one container of mold growth medium; and instructions for using the kit components to detect the presence of mold wherein the kit components are packaged together and sold as a single unit. The kit also includes a mailer to be optionally used if further laboratory analysis of the plates is desired.

In another embodiment, the invention also provides a method for determining the presence or absence of airborne mold spores in multiple locations (i.e., rooms or areas) within a structure, such as a home or an office. The method includes providing a mold test kit having (i) a plurality of culture plates with lids for growing mold and (ii) at least one container of mold growth medium, wherein these components are packaged and sold together as a single unit. The lids are removed from the plates and mold growth medium is poured into each plate. In order to collect representative air samples, the plates are strategically placed throughout multiple locations within a structure and wherein at least one plate is placed outside of the structure (i.e., preferably out doors) as an air control reference plate. It is recommended that the plates be located in a kitchen, family room and main bedroom. Then, an indoor heating and/or a cooling system are turned on and allowed to run for at least about one hour. After one hour, all plates are covered with their corresponding lids and properly labeled. All plates are then placed in a dark, warm location, such as a upper kitchen cabinet, for at least about 48 hours to allow for mold growth. The indoor plates are then compared to the reference plate for the presence of mold growth. The method also including, the option of re-inspecting the plates for mold growth after at least about 72 hours to see if any slow growing molds appear; and if desired submitting the plates for laboratory analysis. To obtain more accurate and reliable test results, it is recommended that all sample collections be obtained simultaneously.

As used herein, the term “mold” refers to a wide range of fungi found virtually everywhere indoors and outdoors, such as for example, hyphal elements, Aspergillus/Penicillium, Epicoccum, Cladosporium, and Phoma species. Generally, Molds produce microscopic cells called spores, which act like seeds to form new mold growths (colonies) when they find the right conditions, such as in dark areas of excess moisture. When molds grow indoors, spores become concentrated in the indoor air, and can cause health problems when they are inhaled in large numbers. It is well known that excess moisture usually occurs in the following areas: air-conditioning units, basements, attics, and crawl spaces; bathrooms; carpets; heating and air-conditioning ducts; humidifiers and dehumidifiers; refrigerator drip pans; and water and sewer pipes. Identifying mold is important because it is a common cause of asthma and/or allergic reaction with sensitized people.

As used herein, the term “mold growth medium” refers to a culture medium (agar or gel) that provides nutrients and moisture for growth of fungal organisms. Many different recipes and formulations are commercially available which include for example, a seaweed derived powder to solidify the mixture. Two common fungal agars include Malt Extract Agar (MEA) and Potato Dextrose Agar (PDA).

As used herein, the term “inspecting” refers to visual examination (i.e., naked eye) of the mold growth on the culture plate. Molds, however, can only be positively identified with a microscope. Bluish-green to green molds usually indicate Penicillium or Aspergillus. Black to brown-black molds can be Aspergillus niger, Alternaria alternata, Cladosporium herbarum, Cladosporium sphaerospermum, or Stachybotrys chartarum (a highly toxic mold). Reddish or pink molds usually indicate species of Fusarium.

### EXAMPLE

In a preferred embodiment, the home mold test kit contains the following items: four 47 mm plastic Petri dishes; one 25 milliliter screw cap bottle of liquid mold growth agar; white labels for use in identifying the location of the air sample; an instruction book for properly using the kit; a “Guide to Mold and Mold Contamination” book; and a mailer, preferably an envelope, to facilitate transport of the culture plates to a laboratory if further analysis of the specific mold type is desired and a clear plastic “clamshell” package housing all of the kit components together.

In a preferred method of practicing the invention, the four pre-treated culture plates are carefully removed from the package containing all of the above-identified kit components and placed on a level surface. The box may be saved for later use. The lids from all four plates are removed. The inside of the plates should not be touched to prevent contamination. Depending on the climate in which the mold test kit is being used, some weather conditions may cause a moisture layer to build up on the bottom of the plates. The layer should not be removed. The droplets do not interfere with the performance of the mold test kit. The bottle containing the mold growth medium (pre-sterilized) should be opened and using a clean, dry teaspoon, one teaspoon of the medium should be poured into each plate. The bottom of each plate should be evenly covered with the medium. The lids should be placed back on the plates, which are to be kept on a level surface until the liquid medium hardens (45 to 60 minutes).

Next, the fan for the HVAC (heating and air conditioning) system should be turned on and allowed to run for about one hour or until a sample collection is finished. The lid from one plate should be removed and the plate placed outside in a protected area on a table or beneath an overhang, away from trees or bushes. This outside plate serves as the control air sample. The control plate should not be located directly in the sun. For best results, the test must not be conducted while it is raining. The three remaining culture plates are to be placed indoors, preferably, strategically located in separate rooms or areas throughout the home or office. To avoid contamination, the lids should be kept on the plates until they have been placed in the desired interior location.

For example, in a home environment, it is suggested that one plate should be placed on a kitchen counter or table; another plate in the living area or family room and the final plate (if available) in the primary bedroom area, or any other area of concern (i.e., where humans and pets will be spending time). Similarly, when testing an office or apartment, if one plate cannot be placed outside, the plate should be placed in an area not directly related to the office/apartment, such as a hallway. The remaining plates should be placed throughout the office/apartment, particularly in any areas of concern.

Once the culture plates are placed in their desired locations, the lids should removed. The plates are to be exposed to the air in the room or area to be tested for about one hour. After one hour, the lids are to be put back on the plates. The test locations should be written on the labels provided, and attached to the bottom of each testing plate. All four covered plates are to be placed in a dark, warm place (such as a kitchen cabinet) for forty-eight hours. After forty-eight hours, the plates are then visually inspected for mold growth. If there is no mold growth, the plates should be inspected after seventy-two hours to be sure no slow-growing molds were missed.

If the plates in one or all of the plates have more mold growth than the outdoor reference plate, that is an indication that mold is growing in the house or office and mold spores are being released into the air. It may be desired to further identify the specific type of mold(s) that are presence in the air and to do this a mailer is provided in the kit to facilitate transport of the culture plates to a laboratory for further analysis. Accordingly, further action, such as mold removal and remediation may be required.

In a related embodiment of the invention, if there is a visible mold problem, one plate should be used to test a sample of the visible mold. To test the visible mold on a wall or other location, the tip of a clean cotton swab should be wiped across the visible mold growth. The mold sample is to be gently wiped from the swab onto the gel in the plate. The plate should be covered and the location identified on a label, which is preferably to be placed on the outside bottom of the plate for easy identification.

It is understood that certain adaptations of the invention described in this disclosure are a matter of routine optimization for those skilled in the art, and can be implemented without departing from the spirit of the invention, or the scope of the appended claims.

We claim:

1. A mold test kit, the kit comprising:
a) a plurality of culture plates for sample collection wherein at least one of the plates is a reference;
b) at least one container of mold growth medium; and
c) printed instructions for using the kit components to facilitate mold detection, wherein components (a), (b) and (c) are packaged together as a single unit.

2. The kit of claim 1, further comprising:
d) a plurality of labels for indicating the plate location where the sample was collected; and
e) a mailer to be optionally used for mailing the plates to a laboratory for further analysis.

3. The kit of claim 1 wherein the plates are 47 mm plastic Petri dishes.

4. The kit of claim 1 wherein the container is a 25 milliliter screw cap bottle.

5. The kit of claim 1 wherein the medium is liquid mold growth agar.

6. The kit of claim 1 wherein the sample is an air sample.

7. The kit of claim 1 wherein the air sample contains airborne mold spores.

8. The kit of claim 1 wherein the air sample is collected indoors, inside a structure, preferably in a home or an office.

9. The kit of claim 1 wherein at least one reference plate is placed outdoors, preferably outside the structure.

10. The kit of claim 1 wherein the presence or absence of mold is identified in at least about forty-eight hours.

11. A method for simultaneously determining the presence or absence of airborne mold spores in multiple locations within a structure, the method comprising the step of:
providing the mold test kit of claim 1.

12. A mold test kit, the kit comprising:
a) four 47 mm plastic Petri dishes;
b) one 25 milliliter screw cap bottle of liquid mold growth agar; and
c) printed instructions for using the kit components to facilitate mold detection, wherein components (a), (b) and (c) are packaged together as a single unit.

13. A method for determining the presence or absence of airborne mold spores in multiple locations within a structure, the method comprising the steps of:
a) providing a mold test kit including (i) a plurality of culture plates with lids for growing mold, and (ii) at least one container of mold growth medium, wherein the kit components (i) and (ii) are packaged together as a single unit;
b) removing the lids from the plates;
c) pouring mold growth medium into each plate;
d) collecting air samples by placing the plates in multiple locations within the structure, wherein at least one plate is placed outside of the structure to serve as a reference plate;
e) running a heating and/or a cooling system within the structure for at least about one hour;
f) covering all plates with the corresponding lids and labeling each plate;
g) placing all plates in a dark, warm location for at least about 48 hours; and
h) inspecting the plates for mold growth.

14. The method further comprising:
i) optionally re-inspecting the plates for mold growth after at least about 72 hours;
and
j) submitting the plates for laboratory analysis.

15. The method of claim 13 wherein the structure is a home or an office.
